(19) United States
(12) Patent Application Publication
(10) Pub. No.: US 2006/0073170 A1
(43) Pub. Date: Apr. 6, 2006

(54) Vaccine complex for preventing or treating leishmaniases

(75) Inventors: Gerard Papierok, Hyeres (FR); Serge Vicens, Gignac la Nerthe (FR); Jean-Loup Lemesre, Montpellier (FR)

Correspondence Address: EGBERT LAW OFFICES, 412 MAIN STREET, 7TH FLOOR, HOUSTON, TX, 77002, US

(73) Assignee: BIO VETO TESTS, en abrege BVT (SARL), La Seyne Sur Mer (FR)

(21) Appl. No.: 10/480,026

(22) PCT Filed: May 30, 2002
(86) PCT No.: PCT/FR02/01823

§ 371 Date: Oct. 11, 2005

(30) Foreign Application Priority Data
Jun. 11, 2001 (FR) 01/07606

(51) Int. Cl. A61K 39/002

(52) U.S. Cl. 424/269.1

(57) ABSTRACT

A therapeutic vaccine complex for preventing or treating leishmaniases and infections mediated by intracellular pathogenic micro-organism in mammals and in particular in humans, members of the dog, car and horse family. The invention is characterized in that it includes excretion secretion molecules derived from Leishmania sp. Promastigotes produced in a specific germ-free and serum-free medium.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention involves a specific immunomodulator complex comprised of excretion-secretion antigens from Leishmania and its use in infections by pathogenic intracellular microorganisms in mammals and in particular, in humans, canines, felidae, and equidae.

More specifically, the present invention involves a therapeutic vaccine complex designed for the prevention and treatment of leishmaniases and infections by pathogenic intracellular microorganisms in mammals and in particular, in humans, canines, felidae, and equidae, whereby this vaccine complex is notably characterized in that it is comprised of excretion-secretion molecules coming from amastigotes and/or promastigotes of Leishmania sp. produced in a specified axenic or aserumal medium.

### BACKGROUND OF THE INVENTION

The leishmaniases comprise a group of parasitic endemic, or even epidemic, infections of the tropical and subtropical regions of the world. The leishmania, flagellate protozoans of the family Trypansomatidae and the genus Leishmania, are the pathogenic agents responsible for these diseases. These parasites affect numerous species of mammals, among which humans and dogs comprise the principal domestic reservoirs of these diseases. The leishmanias are transmitted to these different hosts during the infecting bite of a small midge, called a phlebotomy. Nineteen species of leishmanias are potentially capable of infecting humans, and depending on the species of leishmanias involved, and factors peculiar to the host (genetic, immunological . . . ), they are the source of very diverse clinical manifestations. They develop mainly into three distinct clinical forms: cutaneous, mucocutaneous, and visceral depending on whether the parasites affect the mononuclear phagocytic system of the dermis, the mucous membranes or the internal organs. The cutaneous lesion can remain localized at the point of inoculation of the parasite and correspond to a benign form with spontaneous healing. Besides this form, more serious pathologies exist, caused by disseminated cutaneous leishmaniases and mucocutaneous leishmaniases which are very mutilating and disfiguring.

Visceral leishmaniasis affects the mononuclear phagocytic system of numerous organs and tissues, notably the liver, the spleen, and the bone marrow (hepatomegaly and splenomegaly) and is fatal in the absence of treatment.

As for all vector transmitted diseases, leishmaniases are characterized by a life cycle that is relatively simple since it is divided between two hosts, mammalian and phlebotomic, and consists of two main forms:
a flagellate form called a promastigote, present in the digestive tract of the phlebotomic vector, where it multiplies prior to acquiring its form that is infectious for the mammalian host, also called the metacyclic form;
a non-flagellate form called amastigote, present in the mammalian host such as dogs and humans.

The phlebotome lives in hot regions of the world (hot Mediterranean or tropical climate). To develop, it requires a temperature greater than 17° C. (ideal conditions between 22 to 25° C.), a humid atmosphere, and the absence of wind.

Suburban zones of the Mediterranean countries, where the presence of dogs is more sizeable, combine suitable environmental conditions so that the phlebotomes can reproduce (manure heaps, farms, gardens, wooden shelters, walls, watered lawns, etc.), which promote a larger density of insects near domestic dogs and humans.

Today, leishmaniases represent a significant public health problem particularly in developing countries, and they are a subject of study and research both fundamental and applied in particular in the field of immunoprophylaxis. Ninety-seven countries spread over4 of the 5 continents are affected by leishmaniases. Threatening some 380 million people throughout the world, these parasitoses affect approximately 18 million people in the world, with approximately 2 million new cases per year, 90% of these cases being recorded in India, Sudan, and Brazil. Fifteen years ago, the annual global frequency was estimated to be 400,000 cases [300,000 cases of cutaneous leishmaniasis (CL) and 100,000 cases of visceral leishmaniasis (VL)], with a general incidence of 12 million clinical cases, and a population at risk of approximately 350 million individuals. Currently, the annual global frequency is estimated to be between 1.5 and 2 million new cases per year, of that, 1 to 1.5 million cases of CL and 500,000 cases of VL.

Whereas the tropical and subtropical populations are on the front line facing these diseases, the risks of canine and human infection in the Mediterranean basin are often underestimated. Visceral leishmaniasis by Leishmania infantum is largely expanded over the different continents of the Old World, and is present everywhere surrounding the Mediterranean basin, the south of France comprising one of the focus areas. Though the vector as well as the parasite present in the south of France appear better adapted to dogs than to humans, the number of human cases of leishmaniasis, currently estimated to be a hundred cases per year, has been growing fast for 10 years and is being further increased by the number of immunodepressed subjects.

Leishmaniasis is also considered to be one of the opportunist diseases of AIDS. Approximately 1500 cases of HIV/Leishmania co-infection are counted in the south of Europe which represents 90% of the reported cases in the world, and Spain is the country the most affected with approximately 60% of these cases.

The domestic dog is the main reservoir of the parasite. Canine leishmaniasis, which is a common pathology of the areas surrounding the Mediterranean, manifests itself in various clinical forms which often lead to the death of the animal. The prevalence of canine leishmaniasis can reach 30% of the canine population in some peripheral urban zones. According to Berrahal et coll. (Am. J. Trop. Med. Hyg. 1996, 55, 273-277), 85% of dogs are PCR (Polymerase Chain Reaction) positive in the endemic zone.

At present, there are no effective immunoprophylactic means against these diseases. The treatment of leishmaniases calls for some available molecules: pentavalent antimony, pentamidine, pyrazolopyrimidines, amphotericin B, aminosidine. Today, a consensus seems to be becoming established in considering the combination of antimony salts-pyrazolopyrimidines as the treatment of choice for canine leishmaniasis. Nevertheless, the dogs under treatment remain infectious, in spite of the apparent clinical healing of the animal.

This means that the symptomatic improvement is not correlated to significant reduction of the parasitic load and that there is an epidemiological risk even if clinical healing continues. This situation is further complicated by the emergence of chemoresistance phenomena.

At the present time, although the problems of chemoresistance considerably complicate treatment, it is still not possible to determine its prevalence in an endemic zone and to diagnose it among patients. Similarly, the molecular bases of this resistance induced in the medically important stage of the parasite (e.g. amastigote) are still not known.

Finally, the cases of co-infection AIDS/leishmaniasis pose a serious public health problem to the extent that the available therapeutics are less effective among persons sick with AIDS as well as any immunodepressed person.

Today, no effective vaccine is currently available to combat these diseases and their control must be done by chemotherapy. Chemotherapy is unfortunately jeopardized by long, toxic and costly treatments accompanied by numerous cases of relapse and by the emergence of chemoresistance phenomena. Today, it appears evident that the treatment of these parasitic diseases over the long term will depend on the discovery of new therapeutic targets and/or vaccines.

### BRIEF SUMMARY OF THE INVENTION

The present invention proposes a therapeutic vaccine complex specific to Leishmania acting on the immune response of the infected mammalian host.

Numerous studies concerning the immune responses during experimental murine leishmaniases have led to the demonstration of the predominant role of cell-mediated immunity and the existence of a duality of the immunological response. There are fundamentally two types of responses against leishmanias: one describes the “sensitivity”, the other describes the “resistance”. The different subpopulations of T lymphocytes (CD4+) limit or exacerbate the infection by means of the lymphokines they secrete. It has thus been demonstrated that the subpopulation of auxiliary T lymphocytes of the Th1 type (producer of interferon gamma and interleukine 2) was capable of eliminating the amastigote intracellular forms by means of the activation of macrophages (Reiner S. L et al., Annu Rev Immunol, 1995, 13, 151-177. Review). Conversely, the subpopulation of auxiliary T lymphocytes of the type Th2 (producer of interleukine 4) is responsible for exacerbating the disease.

In humans, certain facts are comparable by nature. In the dog (natural “reservoir” receptive host in the life cycle of L. infantum), the duality of the immunological response is likely. Only one study led by Pinelli et al. (Infect. immun., 62:229, 1994) on experimental animals naturally infected by L. infantum, made it possible to show that the asymptomatism of the dog (clinical state frequently encountered) is accompanied by the absence of a humoral response and the development of a cell-mediated immunity of the Th1 type with a hypersensitivity reaction of the positive delayed type and elevated rates of interleukine 2 and cachectin (TNF-α) circulating in the biological liquids.

A good vaccine candidate must thus match one or more strongly immunogenic parasitic antigens capable either of blocking the differentiation of the Th2 lymphocytes (Gurunathan S et al., J.Exp Med, Oct. 6, 1997 186, 1137-1147) (mode of intervention comparable to “desensitization” treatments currently practiced in cases of allergy), or promoting the emergence of the Th1 lymphocytes ensuring the implementation of a protective immunity.

The present invention involves a therapeutic and preventative anti-Leishmania vaccine complex that has a vaccinating power linked to the presence of excretion-secretion antigens specific against Leishmania by activation of the Th1 method.

Planning to vaccinate against leishmanias is still problematic today. The attempts have been numerous, but the results are weak and/or contradictory. It can be cited the use of living parasites, irradiated parasites, and completely killed parasites (Moreau Y et coll., 1994, Médecine et Armées, 22, 1, 89-93) which have given variable levels of protection among mice and humans.

In the 1980s, purified extracts of parasitic antigens were used in dogs in inducing an exacerbation of the disease: LIF2 fraction and anti-idiotypical vaccine from Dr. Montjour's team. (CHAUVY, J “Immunotherapy trails on a canine population in an endemic zone of leishmania” thesis no. 36.1993-OGUNKOLADE B. W. et coll. Vet Parasitol, 1988, 28,33-41). Other antigens such as membrane antigens GP63 and lipophosphoglucane (MOREAU Y et coll, Médecine et Armées, 1994, 22, 1, 89-93) have not produced a satisfactory result. Currently, several molecules are in trials and a final result is pending. One cites the heat shock protein HSP83 of Leishmania major which stimulates the Th1method and the protein DP72 (JAFFE. C et al, J of Immunol, 1990, 144, 699-706). However, none of the current immunization protocols make it possible to obtain a sufficient level of protection or in any case, it is not reproducible.

To date, no work has been done with the excretion-secretion antigens of Leishmania.

### BRIEF DESCRIPTION OF THE SEVERAL VIEWS OF THE DRAWINGS

FIG. 1

FIG. 2

FIG. 3

FIG. 4

FIG. 5

FIG. 6

FIG. 7

### DETAILED DESCRIPTION OF THE INVENTION

The present invention comprises an immunomodulator complex that uses these excretion-secretion proteins with an adjuvant inducing either an immunostimulation of the lymphocytary system T of the type Th1 in a reproducible manner, or an immunomodulation of lymphocytes of the type Th2 into a type Th1.

Among the numerous parasites such as Plasmodium, Babesia, Trypanosoma, Toxoplasma, or Shistosoma, it has been shown that excretion-secretion antigens (ESA) play a predominant role in the establishment of the immune response of the host. The ESAs of leishmanias appear to be involved in the penetration of the macrophage by the parasites, in the inhibition of proteolytic lysosomal enzymes of the macrophage and the negative regulation of the molecules of the major histocompatibility complex (Alexander and Russel, Adv. Parasitol., 1992, 31, 175-254). Moreover, some vaccine approaches conducted in the mouse using ESAs have already been planned with success in different parasitoses (Ouaissi et al, Parasitology, 1990, 100, 115-24; James et al., Trans. R. Soc. Trop. Med. Hyg., 1989, 83, 67-72; Précigout et al, Infect. Immun., 1991, 59, 2799-805; Darcy et al., Ann. Biol. Clin., 1989, 47, 451-7; Capron et al., Mem. Inst. Oswaldo Cruz., 1995, 90, 235-4).

But the difficulty in preparation and the numerous serumal and/or cellular contaminants contained in the supernatants of the culture make their use difficult in vaccination.

The culture medium described in the Patent Application of the invention WO 94/26 899 makes it possible to partially solve these problems and to use an abundant source, clean and less costly, of ESAs of the main parasitic stage of leishmanias.

In order to obtain a good yield of the cultivation of promastigotes and amastigotes of leishmanias, the medium of patent WO 94/26 899 has been modified as follows:
For the cultivation of the amastigotes, the addition of sulfurated compounds such as L-cysteine and/or nutrient products such as bathocuproine sulfonic acid has been omitted.

The base medium MA1 with the sulfurated compounds is as follows:

| Components | Quantities for 800 ml | |
|---|---|---|
| Base medium | | |
| medium 199 H ® (×10) (with Hanks | 100 | ml |
| salts)* | | |
| Trypto-caseine soybean ® | 5 | g |
| NaHCO3 | 0.35 | g |
| L-glutamine | 0.75 | g |
| HEPES | 5.95 | g |
| D(+) glucose | 2.50 | g |
| H₂O | Q.S. 800 | ml |
| 199 H medium modified ® | 4 | ml (5%) |
| (×10)** | | |
| Additives | | |
| Bovine hemine | 0.009 | mM |
| Reduced glutathion | 0.08 | mM |

| | |
|---|---|
| Vitamin solution | 2% |
| (×100) | |

To 1000 ml of medium MA1, L-cysteine (3 mM) and bathocuproine sulfonic acid (0.01 mM) is added.

The medium MA1 m (m for modified) is the medium mA1 without L-cysteine and without bathocuproine sulfonic acid, on the other hand, bovine hemine has been replaced by porcine hemine irradiated at 25 kilogray, at a markedly lower concentration (0.003 mM).

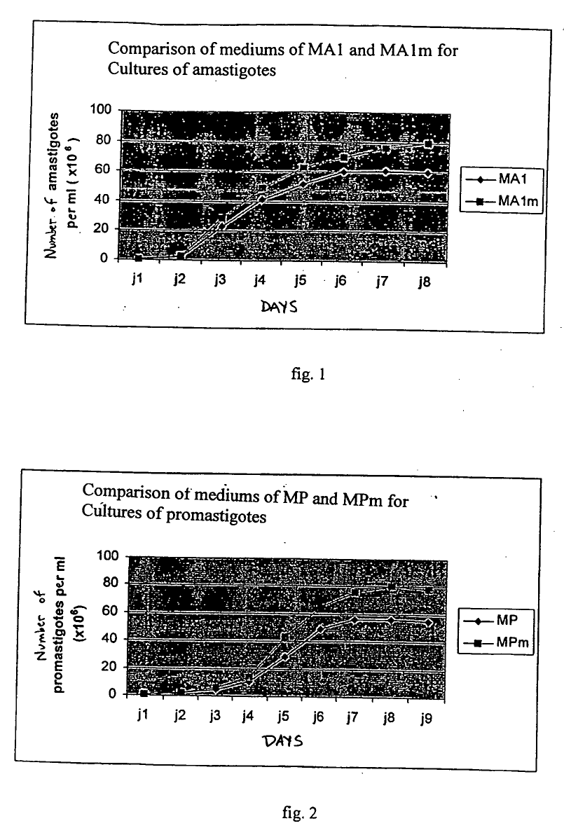

The media MA1 and MA1 m were sown with a strain of Leishmania infantum MON1 and a comparison of the growth of the amastigotes was done at the time (see FIG. 1).
For the cultivation of the promastigotes, a reduction in the concentration of 2 components (RPMI and hemine) as well as the addition of an antibiotic (gentamicine) comprise the main modifications of the reference medium.

It must be added that the bovine hemine has been replaced by porcine hemine irradiated at 25 kilogray as in the case of the modification of the medium for amastigotes.

| | | | Medium MPm (modified) | | |
|---|---|---|---|---|---|
| Medium MP for promastigotes | | | for promastigotes | | |
| RPMI 1640 (1.1×) | 1000 | ml | RPMI 1640 (1×) | 1000 | ml |

| | | |
|---|---|---|
| with L gluthamine and | Medium 199 H modified 10× | 2% |
| Hepes | Porcine hemine irradiated | 0.0002% |

| | | | | |
|---|---|---|---|---|
| Medium 199 H modified (10×) | 2% | Gentamicine sulfate | 0.04 | mg |
| Bovine hemine | 0.0005% | | | |

The media MP and MPm were sown with a strain of Leishmania infantum MON1 and a comparison of the growth of the promastigotes was done at the time (see FIG. 2).

The complex obtained according to the invention comprises molecules naturally excreted by the promastigotes and/or amastigotes of Leishmanias sp., as well as an adjuvant that preferably induces a cell-mediated response.

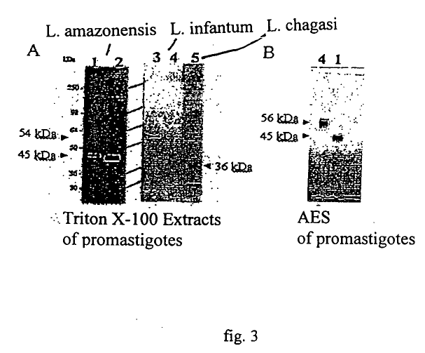

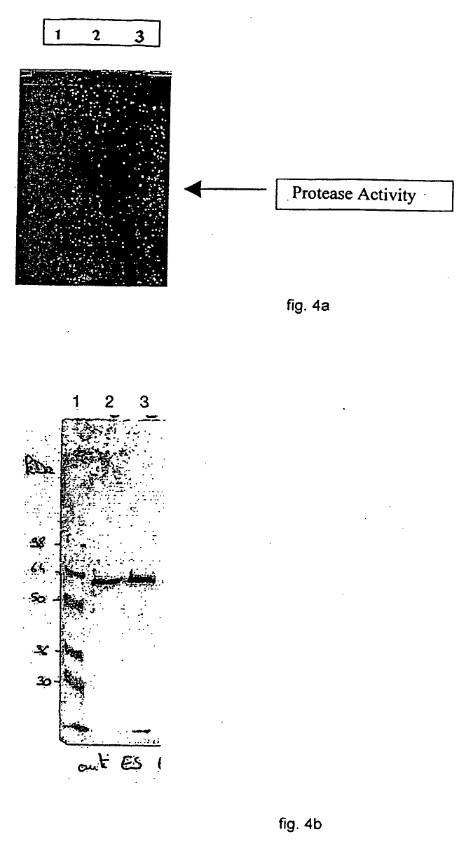

These molecules have at least one common epitope carried by one or more major proteins. Their molecular weight varies from 32 Kda to 200 Kda according to the species of leishmanias and as a function of the parasitic stage considered (FIG. 3: detection of a common epitope in various species of Leishmanias by monoclonal antibodies F5. A: Triton extracts×100 of promastigotes, 1 and 2: L. amazonensis (45 kDa), 3 and 4: L. infantum (54 kDa), 5: L. chagasi (36 kDa), B: AES of promastigotes). These native molecules that express proteasic activities (FIG. 4a: proteasic activity (electrophoresis gel including gelatin), 1=vaccine complex control, 2 and 3=studied vaccine complex) that are unidentified (neither metallic, nor serine, nor cysteine protease) are devoid of any serumal or cellular contaminant.

The promastigote or amastigote forms are cultivated in an axenic or aserumal medium completely defined according to the process described in the Patent Application of the invention WO 94/26 899 cited above and the modification made by the applicant.

The cultures are inoculated at the rate of 5.10⁵ parasites per milliliter of culture medium. The parasites, after incubation, are eliminated by tangential filtration against a membrane of 0.16 μ in polyethersulfone and the filtrate is concentrated 100 times by tangential filtration against a filter of 3 kDa in polyethersulfone.

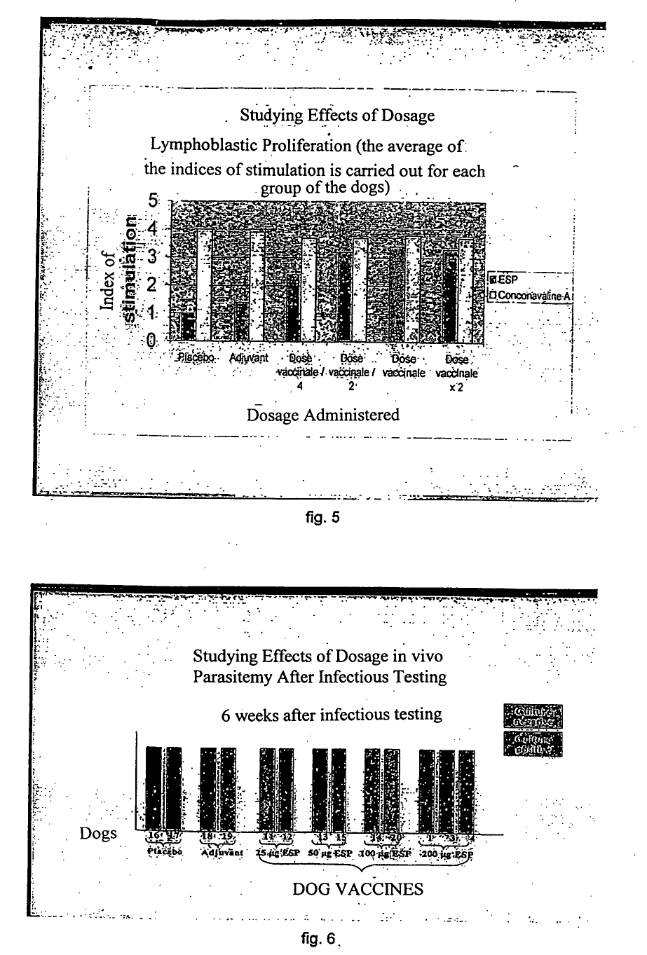

Each lyophilized dose, established according to the dose effect study (FIGS. 5, 6, and 7) is comprised of a lyophilizate of 100 μg of excretion-secretion proteins of Leishmanias and of a diluent comprised of 1 ml of sterile physiological serum.

The composition thus obtained is administered to the infected mammal in the presence of an adjuvant, preferably muramyl dipeptide.

In a preferred manner, the protein/adjuvant ratio is between 1/0.5 and 1/4.

The studies done in dogs have made it possible to determine the optimal vaccine dose to be 200 μg of muramyl dipeptide with a response starting with 100 μg of infected proteins.

The specific action mechanism of the vaccine complex prepared according to the invention is verified using the traditional methods that allow the dosage of the proteins, their identification and the measure of their proteasic activity (techniques of Western Blot or immunoblotting and SDS-PAGE) and using more specific methods that show that the innovative therapeutic vaccine complex acts either by immunostimulation of the lymphocytary system of the Th1 type, or by immunomodulation of the Th2 type towards a Th1 type.

The Western Blot process makes it possible to individually detect proteins, notably excretion-secretion proteins of amastigote (ESA) and excretion-secretion proteins of promastigote (ESP) by antigen/antibody reaction with the corresponding immunoserums.

For each mammal studied (dog for example), a serologic analysis is done with the ESAs and ESPs.

The vaccine complex proteins are separated at first by discontinuous polyacrylamide gel electrophoresis (PAGE) in the presence of sodium dodecyl sulfate (SDS). This separation is followed by an electrophoretic transfer of proteins onto a membrane of nitrocellulose according to the Towbin et al. process (Proc. Nath. Acad. Sci, 1979, 76, 4350-4354). These proteins are then detected by immunoenzymatic reaction by means of an anti-ESP monoclonal antibody (FIG. 4b: Western Blot obtained with a monoclonal anti-AES antibody of promastigotes, 4b1: marker protein (kDa), 4b2: ESP lot to be controlled, 4b3: ESP reference lot).

A parasitologic examination is done on a sample taken directly from the candidate studied, for example, a dog.

A smear of a puncture of the bone marrow is made on a slide. This smear, fixed once by methanol is stained with May-Grünwald-Geimsa and observed by an immersion microscope (×1000).

Samples of the bone marrow are brought under cultivation in the biphase cultivation medium NNN (Novy and Mac Neal, 1904, J. Infec. Dis., 1:1-30), of which RPMI 1640 supplemented with 20% decomplemented fetal calf serum constitutes the liquid phase. The blind subculturings were done every four to six days. The cultures are regularly observed in a photon microscope (×400) for 20 min.

The parasitemy was quantified as follows:

| | |
|---|---|
| +/− | elongated refractive immobile forms; |
| + | 1 to 5 mobile promastigote forms/field; |
| ++ | >5 mobile promastigote forms/field; |
| +++ | culture at confluence. |

Evidence of the Involvement of a Cell-Mediated Immunity of the Th1 Type:

Leishmanias of the promastigote forms are cultivated in the culture media defined according to the methods described above. Parasites are harvested from the end of the exponential phase (6-7 days). The parasitic residue is washed three times by centrifugation (2500 g, 15 mn, 4° C.) in a PBS buffer. After having verified the viability of the parasites using a vital stain (Trypan Blue), a suspension containing 2×10⁸ parasites per ml is inactivated in a PBS buffer containing 0.01% merthiolate (Pinelli et al., 1994, Infect. Immun., 62: 229-235). This constitutes the leishmanias for the intradermoreaction test (JDR).

The study of the immunitary response of the Th1 type that follows was performed on dogs.

Dogs are placed in lateral decubitus and a delicate and non-irritating shearing is done on the thoracic zone approximately 5 cm by 10 cm behind the elbow. Four circles 10 mm in diameter are marked using a felt-tip pen.

Into the center of the circles, 0.1 ml of solution is injected in an intradermo-injection. Two circles receive the solution of leishmanias and the two other circles receive the saline methiolated solution in the negative control. The reading of the Intra Dermo Reaction (IDR) is done 48 hours later using an allergologic gauge.

The test is considered positive if the mean of the two observed induration diameters is greater than or equal to 5 mm. The observation of an erythema without induration will be considered to be a negative test (Pinelli et al., 1994, Infect. Immun., 62: 229-235; Marty et al., 1994, Trans. Roy. Soc. Trop. Med. Hyg., 88, 658-659).

Next, a test is done on the lymphocytary proliferation.

The peripheral blood mononuclear cells (PBMC) of dogs are separated on the Ficoll gradient (density 1,078) by centrifugation at 800 g for 20 mn at ambient temperature. These cells are brought under cultivation on a plate having 96 wells at a concentration of 2.10⁵ cells per well in the presence of 2 μg per ml of Concanavalin A (Sigma), 5 μg per ml of ES P or 20 ml of supernatants of culture harvested in the stationary phase of growth of promastigotes (SP) per well, and in the absence of any additive in a volume of 200 ml of the medium RPMI 1640 supplemented with 5% decomplemented fetal calf serum, 2 mM of L-glutamine, 100 U of penicillin per ml, 100 mg of streptomycin per ml. The optimal antigen and mitogen concentrations have been determined in prior experiments. The PBMCs are incubated for 72 hours in a humid atmosphere at 37° C. in the presence of 5% CO₂ then for 20 hours with 0.5 μCi of ³H thymidine. The cells are harvested over a filter and the incorporation of the radioactivity is determined by counting in a scintillating liquid (β-counter). All of the tests are done in triplicate.

A more rapid and more sensitive immunohistochemical method using BrdU (5-bromo-2′-desoxyuridine), a structural analog of thymidine, is also used to measure the cellular proliferation (BrdU, cell proliferation detection kit III, Boehringer Mannheim, Germany). In our experiments, the BrdU is added for 18 hours after 72 hours of incubation. The cells which have incorporated the BrdU in their ADN are easily detectable in the presence of a monoclonal antibody directed against the BrdU.

The proliferative responses are expressed in stimulation indices that represent the ratio of the average proliferation after stimulation to the mean proliferation in the absence of antigen.

The lymphocytary proliferation has also been estimated by visual readings in a photon microscope (−: negative; ±: slight proliferation; +: little proliferation less than 5 points per microscopic field; ++: mean proliferation greater than 5 points; +++: strong proliferation).

The titration of the leishmanicidal activity of the monocytes is done according to the LEMESRE method described below.

For this test, the monocytes and lymphocytes are isolated from the venous blood of dogs. The monocytes are brought under cultivation for 3 days at the rate of 10⁵ cells per well in the culture chambers (Labteck) in a medium RPMI 1640 complete (containing 25 mM HEPES, 2 mM L-glutamine, 100 U penicillin per ml, 100 mg streptomycin per ml and 10% inactivated fetal calf serum) at 37° C. in a humid atmosphere containing 5% CO₂. After 3 days of cultivation, the macrophages are washed in RPMI medium complete, supplemented with fresh medium and put in contact with the metacyclic promastigote forms of L. infantum in a ratio of 5 parasites per cell, at 37° C. for one night or 5 hours depending on the experiments. The macrophages are then washed with fresh RPMI complete medium in order to eliminate non-phagocytic parasites. The cells are put in incubation either alone, or in the presence of 5 μg of ESP antigens, or in the presence of autolog lymphocytes, or in the presence of supernatants of the co-culture of infected macrophages and autologous lymphocytes and corresponding controls (harvested at 5 hours) and this is done at 37° C. in a humid atmosphere of 5% CO₂ for a duration of 48 hours. When they are used, the lymphocytes cultivated separately are washed, counted, and added to the macrophages in the ratio of 2 lymphocytes per macrophage.

After 48 hours of incubation, the cells are washed three times in a PBS buffer 0.01 M, pH 7.2, fixed in methanol then stained with Giemsa. The leishmanicidal activity of the macrophages is estimated in a photon microscope (1000×) in determining the percentage of macrophages infected and the number of intact amastigote forms for 100 cells (2 times 200 cells are observed in duplicate). The results are expressed in percentage inhibition of the parasitic index=100−(IP×100).
IP=parasitic index=[(mean number of amastigotes per macrophage in the treated sample)×(mean percentage infected macrophages in the treated sample)]/[(mean number of amastigotes per macrophage in the control sample)×(mean percentage infected macrophages in the control sample)].

One can also carry out dosage of nitrogen monoxide (NO) to know the destructive activity of the monocytes against the Leishmanias. The synthesis of NO by the monocytes is in fact a sign of the destruction of the leishmanines by the monocytes having been activated by the cytokines of the interferon gamma type (IFNγ).

NO has a high chemical reactivity. In the presence of water and oxygen, this molecule is rapidly oxidized in a stochiometric manner and forms the nitrites (NO2−) according to the reaction:
4 NO^{°}+O₂+2H₂O - - - 4 NO₂⁻+4 H⁺

The nitrites accumulate in the media and are easily detectable chemically by the Griess method.

To 50 μl of supernatant to be tested, 60 μl of Griess A (sulfanilamide 1% in HCl 1.2 N) is added and 60 μl of Griess B (N-(1-napthtyl)ethyl-enediamine 0.3%) is added. The colorimetric reaction develops in the dark for 2 minutes. The optical densities obtained at 540 nm are corrected by the subtraction of the OD obtained on the wells containing only the culture medium.

The values obtained are recorded on a calibration curve (OD=f(NO₂) made from the known concentrations of NO₂⁻.

The table below shows the serologic responses obtained during our experiments and the monitoring of the parasitemy (analyses made 2 months and 8 months after the infectious test).

| | | | | | | PARASITEMY | |
|---|---|---|---|---|---|---|---|
| | | SEROLOGY | | | | (on marrow puncture) | |
| | | | | | ELISA | | Cultivation |
| | | IF | WB | WB | (IgG2) | Direct | on NNN |
| | Dogs | quantitative | (ESA) | (ESP) | ESA/ESP | Exam | medium |
| Dogs | MUMA | − | − | − | − | + | ++ |
| Controls | LEO | − | − | − | − | + | ++ |
| Dogs | LOUBARD | 1/200 | + | + | +(0.700) | − | − |
| Immunized | MINA | 1/200 | ± | ± | +(0.450) | − | − |
| ESA | | | | | | | |
| Dogs | NOUGAT | 1/800 | + | + | +(0.780) | − | − |
| Immunized | MINON | 1/100 | ± | ± | +(0.520) | − | − |
| ESP | | | | | | | |

Key:
IF: Immunofluorescence (considered positive if the titer is ≧ 1/100)
WB: Western Blot
ELISA: Cut off = 0.300 OD (optical density)
Parasitemy: cultivation on medium NNN
− = absence
++ = more than 5 mobile promastigote forms/field

The following table shows the cellular type responses obtained and the inhibitor role of the serums on the parasitic proliferation (analyses made 2 months after the infectious test).

| | | | | | | PERCENTAGE INHIBITION | |
|---|---|---|---|---|---|---|---|
| | | | | | | OF THE LEISHMANIAS | |
| | | CELL-MEDIATED RESPONSES | | | | PROLIFERATION | |
| | | | Test for | Leishmanicidal | Dosage | Proliferation of | Proliferation of |
| | | | lymphoblastic | activity of the | of NO | the | the |
| | Dogs | IDR | proliferation | monocytes | (in μM) | promastigotes | amastigotes |
| Dogs | MUMA | + | +2.1 (3) | 15.5% | 0.3 | 20% | 15% |
| Controls | LEO | − | +1.2 (3.1) | 21.5% | ND | ND | ND |
| Dogs | LOUBARD | + | ++2.9 (3.2) | 58.9% | ND | 50% | 41% |
| Immunized | MINA | + | ++3.8 (4.2) | 47.8% | ND | 69% | 52% |
| ESA | | | | | | | |
| Dogs | NOUGAT | + | ++3.1 (4.2) | 75.6% | 3.9 | 98% | 54% |
| Immunized | MINON | + | +++3.5 (4.5) | 64.1% | 2.8 | 72% | 56% |
| ESP | | | | | | | |

Key:
IDR: The Intra Dermo Reaction test is considered positive (+) if the induration is ≧ 5 mm 48 h. after intradermoinjection
Lymphoblastic proliferation test: The results are expressed by a reading in photon microscope and in stimulation indices (between parentheses, stimulation index of the control + Concanavaline A)
+: small proliferation
++: medium proliferation
+++: strong proliferation
Leishmanicidal activity of the monocytes: expressed as a percentage of inhibition of the parasitic index
Dosage of NO:
Inhibitor role of the serums: results expressed as a percentage inhibition of growth
ND = Not determined

Inhibitor Role of the Excretion-Secretion Antibodies Anti-Factors on the Parasitic Development of L. infantum:

These tests intend to show the possible inhibitor effect of the anti-ES antibodies on the proliferation and differentiation in vitro of the parasites.

100 μl of immune serum previously inactivated (56° C. for 45 minutes) from different groups of dogs are placed in contact for thirty minutes at ambient temperature with 5×10⁶ metacyclic promastigote forms. Viability tests before and after treatment (see above) were done to establish the percentage mortality. The parasites treated this way are brought under cultivation, either at 25° C. in the RPMI 1640 medium containing 10% FCS (fetal calf serum), or at 37° C. in the MAA/20 medium (10⁶ parasites per ml of medium). The kinetics of proliferation of the promastigote forms and the kinetics of the amastigote forms are established by daily counting of the cells in a photon microscope. The results are expressed in percentage inhibition of growth.

The innovative character of the vaccine complex according to the invention lies not only in the induction of a specific cellular response of the Th1 type, but also in the production of the low antibody rates that are very effective towards the promastigotes and the amastigotes of Leishmania.

For the procedure of the studies, other specific techniques were used.

Method of Infectious Examination

The infectious examination consists in intravenously injecting 10⁶ treated promastigotes in metacyclic phase in the complement of a healthy dog and 5.10⁶ peritoneal macrophages of a healthy dog, infected in vitro by the amastigotes.

The promastigotes and infected macrophages are diluted in sterile physiological serum for a final volume of 1.5 ml. This mixture is made just prior to injection.

Detection of Immunoglobulins of the Type G2 (IgG2) of Dogs, Specifically of the ES

This detection is done by the Western Blot method while using a conjugate anti-IgG2 (immunoglobulins G2) of dog and by the ELISA method according to the microtitration technique of Kweider et al (J. Immunol. 1987, 138, 299).

The vaccine complex according to the invention can be administered in various ways. However, it is administered in a preferred manner in 4 ways:
either by subcutaneous injection
either by intradermal injection
either by intramuscular injection
or orally

Other administration methods can be used, like the parenteral or intravenous method.

In a general manner, a vaccine appears in injectable form comprised of a lyophilizated fraction that is combined with a liquid fraction or diluent. The doses used for prevention and immunotherapy are different, and are also different depending on the mode of injection:

Sub-Cutaneous and Intramuscular Method
injection of a dose (100 μg of excreted-secreted proteins and 200 μg of adjuvant) in dogs regardless of race, age, and sex for a preventative effect).
injection of half doses (50 μg of excreted-secreted proteins and 100 μg of adjuvant) for immunotherapy of leishmanian dogs.

Intradermo Method
injection of a half dose in dogs for a preventative effect
injection of a quarter dose in leishmanian dogs for a therapeutic effect.

The methods of injections are continued in the examples of immunotherapy and vaccination as well as in the studies of harmlessness (innocuousness).

The harmlessness studies on the vaccine complex were performed on 30 dogs.

All the dogs are adult beagles from 1 year to 6 years, 50% male and 50% female, coming from the non-endemic zone. These dogs are perfectly healthy, have a serology and a Intra Dermo Reaction (IDR) test negative with regard to Leishmania.

Among these dogs, some receive placebos. Parallel to the clinical monitoring, a monitoring of the specific immunitary status with regard to the vaccine complex was performed (demonstration of the induction of the humoral and cell-mediated immunity of the Th1 type, only in vaccinated dogs).

Procedure of the Tests

The tests were done with Good Laboratory Practices (GLP) and Good Clinical Practices (GCP).

| | | | | | |
|---|---|---|---|---|---|
| (Primo vaccination) | → 4 weeks of | → Dose | → 4 weeks of | → Overdose | → 4 weeks of |
| | observation | Repeated | Observation | (2 simultaneous | observation |
| | | | | doses) | |

The doses are injected subcutaneously.

A monitoring of the tolerance is done:

After administration, a direct visual examination: pain, tumefaction, heat, and pruritus is done every day for 14 days from the point of injection.

A monitoring of the general tolerance is also performed. This involves a rapid daily examination with taking of the temperature, a weekly clinical veterinary examination including a ganglionic palpation (popliteal), an abdominal palpation, monitoring for arthritis and uveitis and weighing.

The hematological and biochemical monitoring (creatinine, urea, transaminases) are done 3 weeks after each vaccine injection.

Harmlessness (Innocuousness) Results

Among the 30 dogs, including 7 placebos, no general disorder was observed. Only a few local minor reactions are to be reported: slight edema at the point of injection, moderate erythema and slight pruritis. These problems are benign and spontaneously retrocedent in 24 to 48 hours. They are quite logical for a cell-mediation vaccine.

No anomaly is to be noted at the level of the hematological and biochemical monitoring. Similar results were obtained after intradermal injection in 5 dogs and after intramuscular injection in 5 dogs.

The vaccine complex thus does not have any problem of harmlessness.

Specific Activation of the T lymphocytes of the Th1 Type

In parallel to the serological monitoring by traditional immunofluorescence using strips coated with promastigotes (serological reference method for canine leishmaniasis) which turns out to be low in all of the dogs, the study of the cell-mediated response, by the lymphoblastic proliferation test and by study of the leishmanicidal activity of the monocytes, was done on the 30 dogs.

The 7 placebos did not induce a cellular response specific to the vaccine antigen, on the contrary, the 23 dogs vaccinated indeed have an induction of the Th1 system with notably the lymphocytary proliferation indexes specific to the vaccine complex comparable with the control index (Concanavaline A), which accompanies elevated parasitic inhibition percentages (>40% with a mean of 60% on 23 vaccinated dogs).

The vaccine complex thus has the effect of activating the monocytes towards leishmania by the intermediary of the lymphocytes, while having no effect on the Th2 system.

Dose Effect Study of the Vaccine Complex

This experiment had the goal of determining the minimum vaccine dose which induces an effective Th1 response.

For this purpose, 12 adult beagle dogs from 1 to 6 years old from the non-endemic zone are divided into 6 groups of 2:

| | |
|---|---|
| 1^{st} group: | placebo |
| 2^{nd} group: | placebo + 200 μg of adjuvant |
| 3^{rd} group: | 25 μg excretion-secretion proteins and 50 μg of adjuvant |
| 4^{th} group: | 50 μg excretion-secretion proteins and 100 μg of adjuvant |
| 5^{th} group: | 100 μg excretion-secretion proteins and 200 μg of adjuvant |
| 6^{th} group: | 200 μg excretion-secretion proteins and 400 μg of adjuvant |

These tests are done with GLP and GCP.

| | | | | |
|---|---|---|---|---|
| Primo | 4 weeks | 2^{nd} injection | 4 weeks | Infectious |
| Vaccination | | | | Test |

The infectious test consists in infecting the dogs intravenously using promastigotes in metacyclic phase and monocytes infected with amastigotes.

Following the 2^{nd} injection, the study of the immunitary state makes it possible to confirm that the dogs that received the vaccine complex are indeed in Th1 with a beginning of maximum response on a level starting with 50 μg of excretion-secretion proteins injected. This level phenomenon is observed both for the lymphocytary proliferation test and for the monocyte activity.

The graphic in FIG. 5 shows the results given by the dose effect study: lymphoblastic proliferation study according to the injected vaccine dose.

On the other hand, a parasitemic study on the marrow puncture was done 2 months after the infectious test using the culture reference medium NNN (Novy and MacNeal, J. Infect, Dis, 1904, 1, 1-30).

The 4 placebos dogs and a dog having received 50 μg of excretion-secretion proteins have a positive parasitemy.

The graphic of FIG. 6 shows the results given by the dose-effect study: study of the parasitemy, 6 weeks after the infectious test according to the injected vaccine dose.

Specific Antibodies Linked to the Th1 System

As was previously shown, the Th1 system corresponds to a cell-mediated response with an activation of the macrophages via the lymphocyte producers of specific cytokines. This is the main role of the vaccine complex according to the invention. This cellular response is accompanied by a low humoral response that we can easily demonstrate by the traditional method of immunofluorescence using a conjugate anti IgG total marked by fluoresceine.

Nevertheless, certain preliminary work among humans (KAWANO. P et al, Parasite Immunol, 1995, 17, 451-458) and in dogs (NIETO C. G et al, Vet Immunol and Immunopathology, 1999, 67, 117-130) shows that the IgG isotypes would be markers of the immunitary dichotomy Th1/Th2. More specifically, a dog suffering from leishmaniasis with the conclusive clinical signs has a high level of antibodies mainly of the isotype IgG1, while an asymptomatic dog has antibodies specific to the isotype IgG2. The dogs that received the vaccine complex according to the invention have low levels of IgG2 specific to excretion-secretion proteins, which is in keeping with the preferential expansion of T lymphocytes of the Th1 type.

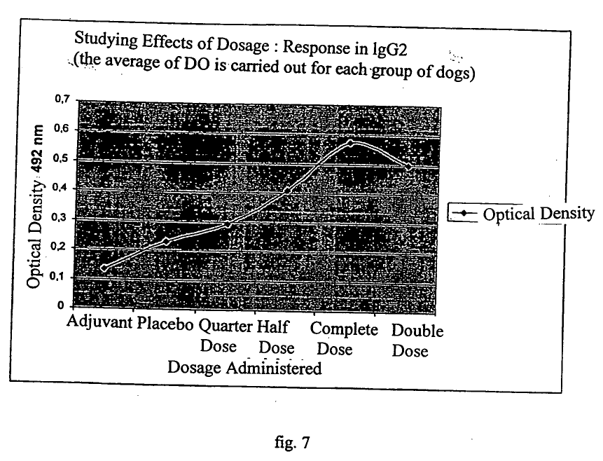

The graphic of FIG. 7 shows the specific response of vaccinated dogs in IgG2 towards the vaccine complex that is the object of the invention, depending on the vaccine dose injected (ELISA method on test wells).

### IMMUNOTHERAPY RESULTS

According to the specialists such as PINELLI (PINELLI. E et al, Infect Immun, 1994, 62, 229-235), the leishmanian dogs correspond to the activation of the lymphocytary system of the Th2 type having a high antibody response.

This increased production of antibodies corresponds to hyperproteinemia and induces the appearance of immune complexes that cause a renal problem (increase in the creatinine and blood urea).

During the studies and trials, an attempt was made to modulate towards a Th1 state by administering to the totally leishmanian dogs intramuscular doses of the vaccine complex. The monitoring of the immune state and the clinical observation were done before and after treatment.

### EXAMPLE 1

### LOYD the Dog

A male dog of the British spaniel breed, named LOYD, age 6 years old, belonging to Ms. C, has numerous cutaneous lesions accompanied with a general state of fatigue and a thin appearance, all reminiscent of a leishmaniasic canine. LOYD lives near Aix-en-Provence in the middle of the endemic zone and spends the majority of his time outside. Thus, he is an animal predisposed to be bitten by the phlebotomes.

The cutaneous lesions are of many types: pustules and papules at the level of the nose; erythema on the side and on the face inside the ears; pruritis, squama and scabs at the level of the elbows.

The veterinarian, Dr. D M, diagnoses a foliaceous pemphigus accompanied by leishmaniasis. This latter diagnosis is confirmed by a direct observation in a microscope of leishmanias from a cutaneous tracing and a serological analysis which gives a titer by immunofluorescence leishmaniasis positive at 1/1600.

For 8 months, a traditional treatment with antimony salts and corticoids turned out to be negative. Then, an immunotherapy was established which consisted in making 4 intramuscular injections of 50 μg of vaccine complex (½ dose), each injection being 10 days apart.

The analysis of the immunitary state prior to any injection makes it possible to confirm that the dog was indeed in an immunitary state of the Th2 type with a high antibody titer as well as negative lymphoblastic proliferation tests and monocytary activation.

A week after the second injection, LOYD the dog regained his appetite and a certain vitality. Dr. D M began to observe a slight cutaneous improvement.

One month after the last injection, LOYD regained anormal clinical appearance with notably an increase in weight of 1 kg and a disappearance of 80% of all cutaneous lesions. Analysis of the immunitary state makes it possible to confirm a reduction in the anti-leishmania antibody titer which dropped to 1/400 by immunofluorescence. In parallel, the monocytes regained a leishmanicidal activity (with a percentage inhibition of the parasitic index equal to 75%) and the lymphoblastic proliferation test is fully positive.

A study of the parasites by cultivation on the NNN medium turned out to be negative. 8 months after treatment, LOYD the dog sometimes had lesions on the nose which correspond to foliaceous pemphigus, lesions disappearing after a corticoid treatment. The biological analyses make it possible to confirm that LOYD is still in an immunitary state Th1.

### EXAMPLE 2

### JAZZ the Dog

A male dog of the Rottweiler breed, named JAZZ, age 5 years old, belonging to Mr. C, has clinical signs specific to leishmaniasis. According to Dr. G H: presence of numerous shiny squama, right periocular hair loss, ulcerous lesions at the level of the 2 front elbows, and a pronounced state of fatigue. Biological analyses with notably a positive leishmaniasis serology at 1/400 by immunofluorescence confirms the clinical diagnostic.

An immunotherapy was established, which consisted in making 3 intramuscular injections of 50 μg of vaccine complex (½ dose), each injection being 10 days apart. The analysis of the immunitary state prior to any injection showed that Jazz the dog had developed an immunitary system of the Th2 type with a greatly positive parasitemy from the bone marrow.

One month after the last injection, the leishmaniasic clinical signs of JAZZ had retroceded with notably a healing of the ulcerous lesions, a sizeable disappearance of the squama and an almost non-existent periocular hair loss. The serology still has a titer by immunofluorescence equal to 1/400. On the contrary, the analysis of the cellular response makes it possible to confirm that JAZZ has an active Th1 state with a positive lymphoblastic proliferation test and a high intramacrophagic leishmanicidal activity.

In parallel, the parasitemy is negative (cultivation of the bone marrow in a NNN medium).

The present invention thus indeed consists of a therapeutic vaccine complex that induces the passage from an immunitary state of the Th2 type, with sizeable production of antibodies that exacerbate the clinical manifestations, to an immunitary state of the Th1 type that leads to healing.

### VACCINATION RESULTS

In order to evaluate the efficacy of the vaccine complex according to the invention, the vaccine complex was tested on 6 perfectly healthy dogs. These 6 dogs have a negative leishmaniasic serology, a negative parasitemy as well as fully negative cellular response tests specific to Leishmania.

These 6 dogs live in a place free from any phlebotomes. We define 3 groups of dogs, each group comprising a male and a female.

Control Group (Placebos)
Negative control the dog named LEO, Pointer breed, male. 3 years old.
Sole adjuvant control: the dog named MUMA, British spaniel breed, female. 6 years old.

Group of Dogs Vaccinated with Excretion-Secretion Proteins of Promastigotes (ESP)
The dog named MINON, Weimaraner breed, female. Age: 2 and a half years old.
The dog named NOUGAT, Pointer breed, male. Age: 2 and a half years old.

Group of Dogs Vaccinated with Excretion-Secretion Proteins of Amastigotes (ESA)
The dog named LOUBARD, British spaniel breed, male. Age: 4 years old.
The dog named MINA, Weimaraner breed, female. Age: 3 years old.

The vaccine injection scheme is as follows:

| | | |
|---|---|---|
| Day 0 | Day 28 | Day 84 |
| 1^{st} injection | 2^{nd} injection | |
| 1 subcutaneous → 4 weeks → | 1 subcutaneous → 8 weeks → | infectious |
| dose | dose | test |

A clinical monitoring of the 6 dogs was done every two weeks. The biological analyses were scheduled as follows:

| | | | | | |
|---|---|---|---|---|---|
| Day 0 | | Day 28 | | Day 84 | Day 84 |
| 1^{st} injection | ↓ | 2^{nd} injection | ↓ | infec- | +2 months |
| | Day 14 | | Day 56 | tious | ↓ |
| | Biological | | Biological | test | Biological |
| | analyses | | analyses | | analyses |

The biological analyses consist of:
biochemical analyses: urea, creatine, transaminases
hematological analyses: count, formula
serology leishmaniasis: quantitative anti-Leishmania immunofluorescence, by the Western Blot method towards excretion-secretion antigens and dosage by the ELISA method of specific IgG2s.
cellular response tests: lymphoblastic proliferation test, study of the activation of macrophages and IDR test (IntraDermoReaction), dosage of NO.
study of the neutralizing role of the anti ES antibodies.

To these analyses must be added the search for leishmania by direct observation in a microscope and cultivation on medium NNN from bone marrow after the infectious test.

Results

Clinical Monitoring.

No significant clinical manifestation appeared during all of this study. A slight weight loss and the appearance of some squama in the dog LEO must be noted 2 months after the infectious test.

Biological Monitoring.

The biochemical and hematological parameters stayed normal all during this study.

Leishmaniasis serology and parasitemy

Prior to any injection, the 6 dogs have negative serologies and parasitemies. The following table shows the serological responses obtained during the experiments performed and the monitoring of the parasitemy (analyses made 2 months and 8 months after the infectious test).

| | | | | | | PARASITEMY | |
|---|---|---|---|---|---|---|---|
| | | SEROLOGY | | | | (on marrow puncture) | |
| | | | | | ELISA | | Cultivation |
| | | IF | WB | WB | (IgG2) | Direct | on NNN |
| | Dogs | quantitative | (ESA) | (ESP) | ESA/ESP | Exam | medium |
| Dogs | MUMA | − | − | − | − | + | ++ |
| Controls | LEO | − | − | − | − | + | ++ |
| Dogs | LOUBARD | 1/200 | + | + | +(0.700) | − | − |
| Immunized | MINA | 1/200 | ± | ± | +(0.450) | − | − |
| ESA | | | | | | | |
| Dogs | NOUGAT | 1/800 | + | + | +(0.780) | − | − |
| Immunized | MINON | 1/100 | ± | ± | +(0.520) | − | − |
| ESP | | | | | | | |

Key:
IF: Immunofluorescence (considered positive if the titer is ≧ 1/100)
WB: Western Blot
ELISA: Cut off = 0.300 OD (optical density)
Parasitemy: cultivation on medium NNN
− = absence
++ = more than 5 mobile promastigote forms/field

Only the immunized dogs have antibodies specific towards ESA and ESP (Western Blot), the specific IgG2s (ELISA) and negative parasitemies. A slight appearance of total antibodies ( 1/200 in IF) must be noted in all of the dogs after the infectious test.

Only the control dogs (LEO and MUMA) have positive parasitemies and an absence of specific antibodies IgG2 anti ES.

Cell-Mediated Response

Before any injection, the 6 dogs had a fully negative cell-mediated response to Leishmania infantum. According to Table II, only the immunized dogs have positive lymphoblastic proliferation tests, intramacrophagic leishmanicidal activities linked to the production of NO by the monocytes.

The following table shows the cellular type responses obtained and the inhibitor role of the serums on the parasitic proliferation (analyses made 2 months after the infectious test).

| | | | | | | PERCENTAGE INHIBITION | |
|---|---|---|---|---|---|---|---|
| | | | | | | OF THE LEISHMANIAS | |
| | | CELL-MEDIATED RESPONSES | | | | PROLIFERATION | |
| | | | Test for | Leishmanicidal | Dosage | Proliferation of | Proliferation of |
| | | | lymphoblastic | activity of the | of NO | the | the |
| | Dogs | IDR | proliferation | monocytes | (in μm) | promastigotes | amastigotes |
| Dogs | MUMA | + | +2.1 (3) | 15.5% | 0.3 | 20% | 15% |
| Controls | LEO | − | +1.2 (3.1) | 21.5% | ND | ND | ND |
| Dogs | LOUBARD | + | ++2.9 (3.2) | 58.9% | ND | 50% | 41% |
| Immunized | MINA | + | ++3.8 (4.2) | 47.8% | ND | 69% | 52% |
| ESA | | | | | | | |
| Dogs | NOUGAT | + | ++3.1 (4.2) | 75.6% | 3.9 | 98% | 54% |
| Immunized | MINON | + | +++3.5 (4.5) | 64.1% | 2.8 | 72% | 56% |
| ESP | | | | | | | |

Key:
IDR: The Intra Dermo Reaction test is considered positive (+) if the induration is ≧ 5 mm 48 h. after intradermoinjection
Lymphoblastic proliferation test: The results are expressed by a reading in photon microscope and in stimulation indices (between parentheses, stimulation index of the control + concanavaline A)
+: small proliferation
++: medium proliferation
+++: strong proliferation
Leishmanicidal activity of the monocytes: expressed as a percentage of inhibition of the parasitic index
Dosage of NO:
Inhibitor role of the serums: results expressed as a percentage inhibition of growth
ND = Not determined
Study of the neutralizing role of the anti-ES antibodies:

This analysis done on 1 dog for each group (Table II) makes it possible to confirm that the dogs immunized by ES have very effective antibodies inhibiting both the proliferation of promastigotes and those of the amastigotes against control dogs.

From these analyses, the vaccine complex indeed induces a cell-mediated immunity of the Th1 protector type, to which it is necessary to add an induction of the antibodies specific to isotype IgG2 significantly inhibiting the proliferation of leishmanias.

1. Therapeutic vaccine complex designed for the prevention or treatment of leishmaniases and infections by pathogenic intracellular microorganisms in mammals and in particular, in humans, canines, felidae, and equidae, characterized in that it is comprised of excretion-secretion molecules coming from promastigotes of Leishmania sp. produced in an specified axenic or aserumal medium.

2. Therapeutic vaccine complex designed for the prevention or treatment of leishmaniases and infections by pathogenic intracellular microorganisms in mammals and in particular, in humans, canines, felidae, and equidae, characterized in that it is comprised of excretion-secretion molecules coming from amastigotes of Leishmania sp. produced in an specified axenic or aserumal medium.

3. Therapeutic vaccine complex designed for the prevention or treatment of leishmaniases and infections by pathogenic intracellular microorganisms in mammals and in particular, in humans, canines, felidae, and equidae, characterized in that it is comprised of excretion-secretion molecules coming from amastigotes and promastigotes of Leishmania sp. produced in an specified axenic or aserumal medium.

4. Therapeutic vaccine complex designed for the prevention or treatment of leishmaniases and infections by pathogenic intracellular microorganisms in mammals, according to one of the claims 1 to 3, characterized in that the axenic and aserumal medium specifically defined corresponds to a modification of the previously known media leading to an improvement of the cultivation of amastigotes and promastigotes of Leishmania sp., by omitting, in the case of cultivation of amastigotes, the addition of sulfurated compounds such as L-cysteine and/or nutrient products such as bathocuproine sulfonic acid and by replacing bovine hemine with porcine hemine at a markedly lower concentration and, in the case of cultivation of promastigotes, by further lowering the concentration of some components (RPMI and hemine) and by adding an antibiotic (gentamicine).

5. Therapeutic vaccine complex designed for the prevention or treatment of leishmaniases and infections by pathogenic intracellular microorganisms in mammals, according to one of the claims 1 to 4, characterized in that the excretion-secretion molecules coming from promastigotes and/or amastigotes of Leishmania sp. have at least one epitope in common carried by one or more major proteins.

6. Therapeutic vaccine complex designed for the prevention or treatment of leishmaniases and infections by pathogenic intracellular microorganisms in mammals, according to one of the claims 1 to 5, characterized in that the molecular weight of the excretion-secretion molecules coming from promastigotes and/or amastigotes of Leishmania sp. varies between 32 and 200 kDa as a function of the parasitic stage and the species of microorganisms under consideration.

7. Therapeutic vaccine complex designed for the prevention or treatment of leishmaniases and infections by pathogenic intracellular microorganisms in mammals, according to one of the claims 1 to 6, characterized in that some of the excretion-secretion molecules coming from promastigotes and/or amastigotes of Leishmania sp. have unidentified proteasic activities (neither metallic, nor serine, nor cysteine protease).

8. Therapeutic vaccine complex designed for the prevention or treatment of leishmaniases and infections by pathogenic intracellular microorganisms in mammals, according to one of the claims 1 to 7, characterized in that the excretion-secretion molecules coming from promastigotes and/or amastigotes of Leishmania sp. are combined with an adjuvant that preferably induces a cell-mediated response.

9. Therapeutic vaccine complex designed for the prevention or treatment of leishmaniases and infections by pathogenic intracellular microorganisms in mammals, according to claim 8, characterized in that the adjuvant is advantageously muramyl dipeptide.

10. Therapeutic vaccine complex designed for the prevention or treatment of leishmaniases and infections by pathogenic intracellular microorganisms in mammals, according to claim 9, characterized in that the muramyl dipeptide is combined with the excretion-secretion molecules coming from the promastigotes and/or amastigotes of Leishmania sp. in a ratio protein/adjuvant of 1/0.5 to 1/4.

11. Therapeutic vaccine complex designed for the prevention or treatment of leishmaniases and infections by pathogenic intracellular microorganisms in canines, according to claims 9 to 10, characterized in that the muramyl dipeptide is combined with the excretion-secretion molecules coming from the promastigotes and/or amastigotes of Leishmania sp. in a ratio of 100 μg of proteins to 200 μg of muramyl dipeptide.

12. Therapeutic vaccine complex designed for the prevention or treatment of leishmaniases and infections by pathogenic intracellular microorganisms in mammals, according to one of the claims 1 to 11, having the particularity of inducing in mammals a leishmanicidal activity of the parasited monocytes that can be measured according to the LEMESRE method for determining the inhibition of the parasitic index at 48 hours of incubation.

13. Therapeutic vaccine complex designed for the prevention or treatment of leishmaniases and infections by pathogenic intracellular microorganisms in mammals, according to any one of the claims 1 to 12, having the particularity of inducing in mammals an activation of cell-mediated immunity dependent on the T lymphocytes and preferentially the T lymphocytes of the Th1 type.

14. Therapeutic vaccine complex designed for the prevention or treatment of leishmaniases and infections by pathogenic intracellular microorganisms in mammals, according to one of the claims 1 to 12, having the particularity of inducing in mammals the passage from an immunitary state of the Th2 type to an immunitary state of the Th1 type.

15. Therapeutic vaccine complex designed for the prevention or treatment of leishmaniases and infections by pathogenic intracellular microorganisms in mammals, according to any one of the claims 13 or 14, having the particularity of inducing in mammals specific antibodies and more particularly antibodies specific to isotype IgG2.

16. Therapeutic vaccine complex designed for the prevention or treatment of leishmaniases and infections by pathogenic intracellular microorganisms in mammals, according to any one of the claims 1 to 15, packaged in a form so that it can be administered in different ways: subcutaneous, intradermal, intramuscular, intravenous, parental [sic—parenteral?] and oral.
